(19) United States
(12) Patent Application Publication
(10) Pub. No.: US 2021/0307624 A1
(43) Pub. Date: Oct. 7, 2021

(54) NON-INVASIVE DEVICE AND METHODS FOR MONITORING MUSCLE TISSUE CONDITION

(71) Applicant: ODIN TECHNOLOGIES, LLC, Chicago, IL (US)

(72) Inventors: Ruchira Pratihar, Houston, TX (US); Paul Hansen, Saratoga, CA (US); Steven Matthew Hansen, Chicago, CA (US)

(21) Appl. No.: 17/210,019

(22) Filed: Mar. 23, 2021

Related U.S. Application Data
(63) Continuation of application No. PCT/US2019/053310, filed on Sep. 25, 2019.
(60) Provisional application No. 62/737,418, filed on Sep. 27, 2018.

(51) Int. Cl. A61B 5/026; A61B 5/00; A61B 5/1455

(52) U.S. Cl. CPC A61B 5/0261; A61B 5/4519; A61B 5/0022; A61B 5/14551; A61B 5/7455; A61B 5/6831; A61B 5/6833; A61B 5/7435; A61B 5/7405; A61B 5/7275

(57) ABSTRACT

Devices and methods are provided herein for monitoring tissue condition. A device as described herein may comprise a base layer configured to attach or encompass a portion of the subject's body in proximity to the target site; a plurality of light emitting diodes (LEDs) coupled to the base layer corresponding to one or more predetermined locations on the portion of the subject's body, wherein the plurality of LEDs is configured to emit light signals at the one or more predetermined locations penetrating beneath the skin of the subject to the target site; and a plurality of photodiodes configured to receive reflected light signals associated with the target site, wherein the reflected light signals are analyzed to determine a plurality of physiological parameters comprising at least volumetric blood flow through, at, or in proximity to the target site, and wherein the plurality of physiological parameters is used to monitor a state of the target site.

### CROSS-REFERENCE

This application is a continuation application of International Application No. PCT/US2019/053310, filed on Sep. 26, 2019, which application claims the benefit of U.S. Provisional Application No. 62/737,418, filed Sep. 27, 2018, which application is incorporated herein by reference in its entirety.

### BACKGROUND

In humans and many other animals, groups of muscles are housed within a protective sheath called fascia. This fascia is a rigid connective tissue that keeps the muscle fibers bound together and separates them from other internal organs. When a muscle begins to expand, either due to vascular injury or over exercise, the fascia doesn't expand to compensate for the progressively increasing size of the muscle. As the muscle continues to expand, the restricting fascia causes an increase in pressure within the muscle compartment, referred to as the intracompartmental pressure (ICP). As the ICP of the muscle compartment rises, it begins to crush the arteries and veins within the surrounding tissue; not allowing an adequate supply of blood and nutrients to reach affected tissues.

If a muscle compartment begins to increase in pressure beyond safe levels, typically referred to as an ICP above 30 mmHg (>30 mmHg), the distal tissues will become ischemic and begin to perish. If identified too late, a subject will experience tissue death; requiring either muscle excision, limb amputation, or in serious cases the subject will expire.

Compartment syndrome is traditionally treated with an emergency fasciotomy; a highly invasive procedure wherein the muscle fascia is cut to allow the pressure to release. These operations generally require multiple long incisions along the affected muscle compartment, have high risks of infection, and are associated with long hospital stays. Without an adequate method of diagnosing compartment syndrome, physicians are often performing emergency fasciotomies on subjects that may or may not have compartment syndrome as a prophylactic treatment.

There are multiple known methods for the diagnosis of compartment syndrome, albeit many of these methods require invasive technologies that are injected directly into the muscle compartment. Many of these technologies are cumbersome to use and require physician oversight, making it very difficult for non-physician healthcare providers to diagnose and monitor compartment syndrome over long periods of time. Because of this difficulty, a need has quickly arisen for a new and efficient method of diagnosing and monitoring compartment syndrome.

### SUMMARY

The present disclosure can address at least the above deficiencies and limitations. Various embodiments of the present disclosure address the demand for accessible, easy to use, noninvasive monitoring systems that are capable of locating and constant monitoring a target site without the need for oversight of a physician.

According to some aspects of the disclosure, a device for non-invasive monitoring of a target site in a subject's body is provided. The device may comprise a base layer configured to attach or encompass a portion of the subject's body in proximity to the target site; and a plurality of light emitting diodes (LEDs) coupled to the base layer corresponding to one or more predetermined locations on the portion of the subject's body, wherein the plurality of LEDs are configured to emit light signals at the one or more predetermined locations penetrating beneath the skin of the subject to the target site; and a plurality of photodiodes configured to receive reflected light signals associated with the target site, wherein the reflected light signals are analyzed to determine a plurality of physiological parameters comprising at least volumetric blood flow through, at, or in proximity to the target site, and wherein the plurality of physiological parameters are used to monitor a state of the target site. In some embodiments, the predetermined locations may comprise three predefined locations. The target site may comprise muscle tissue, arterial structures, and/or venous structures in the neck or an extremity of the subject, and wherein the extremity includes an upper extremity and/or a lower extremity. The state of the target site may be characterized by tissue health or lack thereof at the target site. In some embodiments, the plurality of LEDs comprises at least three LEDs. The light signals of the LEDs may be emitted at three or more different wavelengths. In some embodiments, the one or more predetermined locations are situated adjacent to bony structures of the subject's body, and wherein the bony structures are selected from the group consisting of medial malleolus, lateral malleolus, spinal column, femur, calcaneus, fibula, radius, ulna, scaphoid, and tibia of the subject. The one or more predetermined locations may be situated directly above muscular tissue of the subject's body. The one or more predetermined locations may be situated on a wrist, neck, chest, and/or an ankle of the subject. In some embodiments, the plurality of LEDs and photodiodes are operably coupled to a processor via wired or wireless communications. The plurality of LEDs and photodiodes may be provided as a single unit or component.

In some embodiments, the device may further comprise a processor. The processor may be removable from the device with the device maintaining its operability and functionality. The processor may be located on an external monitoring unit or device. The processor may be configured to receive the reflected light signals from the plurality of photodiodes. The processor may be configured to control the plurality of LEDs to emit the light signals at three or more different wavelengths and various pulsed frequencies. The processor may be configured to control the plurality of LEDs to emit the light signals at two different wavelengths and various pulsed frequencies. The processor may be configured to analyze the reflected light signals to determine the plurality of physiological parameters. In some embodiments, the plurality of physiological parameters further comprises blood oxygen saturation (SPO2) content. In some cases, the plurality of physiological parameters further comprises an intracompartmental pressure (ICP). The plurality of physiological parameters may comprise a calculation for a diameter of an arterial structure. In some embodiments, the one or more predetermined locations may be situated superior to arterial structures. In other cases, the one or more predetermined locations may be situated in proximity to the arterial structures.

The plurality of physiological parameters may be used to determine a risk level of the subject for developing ischemic tissues. In some embodiments, the plurality of physiological parameters may be used to determine a risk level of the subject for developing compartment syndrome.

In some embodiments, the physiological parameters may be used to determine the risk level substantially in real time.

In some embodiments, the plurality of physiological parameters may be used to assess whether the subject has perfusion related complications. The plurality of physiological parameters may be used to monitor the progression or regression of any perfusion related complications. The plurality of physiological parameters may be used to assess whether the subject is at risk of developing perfusion related complications.

In some embodiments, the reflected light signals may comprise photoplethysmogram (PPG) signals.

In some embodiments, the emitted light signals are partially absorbed by the arterial structures, and reflected by the arterial structures as the reflected light signals.

The processor may be configured to analyze the reflected light signals using one or more machine learning algorithms to determine the plurality of physiological parameters.

In some embodiments, the plurality of LEDs and photodiodes may collectively constitute a plurality of sensing channels. The reflected light signals from the plurality of sensing channels may be provided to a multiplexer. In some embodiments, the device may further comprise a multiplexer.

In some embodiments, the base layer may be made of a flexible, biocompatible material. The base layer may be configured to substantially conform, attach, or mount onto the portion of the subject's body. The base layer may further be configured to attach, encompass, or mount onto the portion of the subject's body using at least an adhesive, strap, suction, or electromagnetic mechanism. The base layer may serve as a substrate for supporting the plurality of LEDs and photodiodes. The base layer may comprise a plurality of electrical connections for the plurality of LEDs and photodiodes.

In some embodiments, the plurality of physiological parameters may be configured to be displayed on a graphical user interface and/or indicated through visual, auditory, or tactile stimulus. In some embodiments, a notification module is configured to send one or more alerts or status reports while the target site is being monitored using the plurality of physiological parameters.

Also disclosed herein is a method for non-invasively monitoring a target site in a subject's body, the method comprising: attaching a device to a portion of the subject's body in proximity to the target site, wherein the device comprises a base layer, a plurality of light emitting diodes (LEDs) arranged on or coupled to the base layer to correspond to one or more predetermined locations on the portion of the subject's body, and a plurality of photodiodes; and using the plurality of LEDs to emit light signals at the one or more predetermined locations penetrating beneath the skin of the subject through, at, or in proximity to the target site; using the plurality of photodiodes to receive reflected light signals associated with the target site; and analyzing the reflected light signals to determine a plurality of physiological parameters comprising at least volumetric blood flow through, at, and in proximity to the target site; and using the plurality of physiological parameters to monitor a state of the target site.

Analyzing the reflected light signals may comprise comparing received amplitudes of consecutive PPG signals over different time periods. The amplitudes of consecutive PPG signals of the reflected light may be compared for a same photodiode. In some cases, a strength of the reflected light signals is compared between different photodiodes. The plurality of LEDs may comprise at least three LEDs. In some embodiments, the light signals may be emitted at three or more different wavelengths.

Also disclosed herein is a method for non-invasively monitoring a target site in a subject's body, the method comprising: collecting photoplethysmogram (PPG) signals associated with or in proximity to the target site; analyzing a variation in amplitude of PPG signals over time; using one or more machine learning algorithms to calculate a plurality of physiological parameters comprising at least volumetric blood flow through the target site; and using the plurality of physiological parameters to monitor a state of the target site, wherein the target site comprises arterial or arteriole structures in an extremity of the subject, and wherein the state is characterized by tissue health through, at, or in proximity to the target site.

Also disclosed herein is a device for non-invasive monitoring of a target site in a subject's body, the device comprising: a base layer configured to attach, encompass, or mount onto a portion of the subject's body in proximity to the target site; a plurality of light emitting diodes (LEDs) arranged on or coupled to the base layer to correspond to at least one section of thin tissue on the portion of the subject's body, wherein the plurality of LEDs are configured to emit light signals into at least one section of thin tissue to penetrate the skin of the subject to reach the target site; and a plurality of photodiodes configured to receive reflected light signals associated with the target site,

wherein the reflected light signals are analyzed to determine a plurality of physiological parameters comprising at least volumetric blood flow through, at, or in proximity to the target site, and wherein the plurality of physiological parameters is used to monitor a state of the target site. The state may be characterized by tissue health within, at, and in proximity to the target site.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A schematically illustrates the top view of a device in accordance with some embodiments.

FIG. 1B schematically illustrates the bottom view a device in accordance with some embodiments.

FIG. 1C schematically illustrates the charging station of a device in accordance with some embodiments.

FIG. 2 shows a perspective view of a portion of the body at the lower extremity with a monitoring device, according to one embodiment.

FIG. 3 shows the relative location of sensor arrays with regards to the target site according to one embodiment.

FIGS. 4A and 4B and 4C and 4D schematically illustrate the relative locations of sensor arrays with regards to the predetermined locations, according to one embodiment.

FIG. 5 is a perspective view of a workflow of the system in accordance with some embodiments.

FIG. 6 shows an exemplary embodiment of a system collecting, storing and analyzing the collected data.

FIG. 7 shows an example of the hardware architecture used in some embodiments of the disclosure and the relation between hardware and software architectures.

FIG. 8 shows an exemplary embodiment of a smart device application output.

FIG. 9A is an example of PPG signal in accordance with some embodiments.

FIG. 9B is an example of the area of the PPG curve used to measure the strength of the PPG signal in accordance with some embodiments.

FIG. 10 shows an example of a curve of consecutive signals strengths for right and left leg of a subject in accordance with some embodiments.

FIG. 11A is an example of graphical representation of a subject's volumetric blood flow in response to different stimuli in accordance with some embodiments.

FIG. 11B is an example of graphical representation of a subject's blood oxygenation content in response to a tourniquet being applied in accordance with some embodiments.

FIG. 12 is a perspective view of a block diagram of the artificial intelligence architecture according to some embodiments.

FIG. 13A shows a comparison in the flow measurement between the method of the present disclosure and other invasive methods.

FIG. 13B shows an example of an invasive method of measuring intracomparmental pressure.

FIG. 14 shows an example of the classification of high risk and low risk signals according to some embodiments.

FIG. 15 shows a computer system that is programmed or otherwise configured to implement methods provided herein.

### DETAILED DESCRIPTION

Current systems for monitoring compartment syndrome may utilize invasive methods such as direct injection to the muscle compartment to monitor the state of the tissue. These methods may also require physician oversight, making it difficult to use by non-physician health care providers or the patients. The measurements and monitoring for compartment syndrome need to be repeated over time in order to update information about the progression of the muscle inflammation which causes added discomfort for the patient in invasive monitoring methods. The existing invasive methods may be cumbersome, and may not be capable of monitoring early stages of compartment syndrome.

The embodiments of the disclosure described herein can enable continuous and noninvasive monitoring of one or a plurality of subcutaneous target sites. Aspects of present disclosure may reduce or eliminate the need of physician oversight for the measurements.

The present application generally relates to devices, methods and systems in the field of medical devices. In particular, it relates to devices and methods for noninvasive, portable and/or wearable blood volume measurements. The devices and methods described herein may enable measuring subcutaneous processes, such as blood volume measurements, flow measurements, blood oxygen content measurements and detecting physiological phenomena in a patient's body. The present application may incorporate modalities and/or technologies, such as but not limited to light technology, a two-dimensional echo probe for detecting textures under the skin, sound and/or vibration detection, thermal dilution technology, pressure sensing technology, pulse wave technology and/or stress strain detection technology. The present application may incorporate ways of performing monitoring for humans and/or animals of all ages in various fields, including but not limited to monitoring physiological parameters in muscular tissues, arteries, veins, capillaries, ducts, and/or other anatomical conduits.

A device as described herein can be configured to monitor a target site in a subject's body, the device comprising a base layer configured to attach or encompass a portion of the subject's body in proximity to the target site; a plurality of light emitting diodes (LEDs) coupled to the base layer corresponding to one or more predetermined locations on the portion of the subject's body, wherein the plurality of LEDs are configured to emit light signals at the one or more predetermined locations penetrating beneath the skin of the subject to the target site; and a plurality of photodiodes configured to receive reflected light signals associated with the target site, wherein the reflected light signals are analyzed to determine a plurality of physiological parameters comprising at least volumetric blood flow through, at, or in proximity to the target site, and wherein the plurality of physiological parameters are used to monitor a state of the target site. In some embodiments, the predetermined location is a single predetermined location. In some instances, there may be two predetermined locations. In other instances, there may be three predetermined locations. There may be more than three predetermined locations. The target sites by way of nonlimiting examples may include muscle tissue, arterial structures, and/or venous structures in an extremity of the subject, and wherein the extremity includes an upper extremity and/or a lower extremity.

In some embodiments, light emitting and receiving components such as light emitting diodes (LEDs) and photodiodes may be used, including framework systems for mounting, locating and maintaining a plurality of LEDs and photodiodes such as a sensor array, in contact with an anatomical surface (e.g., skin) of a subject, adjustable sensor array mounting systems, and sensor array interface components providing an interface between a sensor array mounting system and the sensor array. Methods for using the sensor array mounting systems, interface components and/or framework structure, and for adjusting the illumination area of sensor array with respect to a target site are also disclosed. These aspects can optimize the quality of the measurements obtained and improve ease of use for the user or wearer.

The embodiments of the disclosure described herein can enable determining a state of a target site. The state of the target site can comprise the health of the target site or lack thereof as well as trending characteristics of the target site.

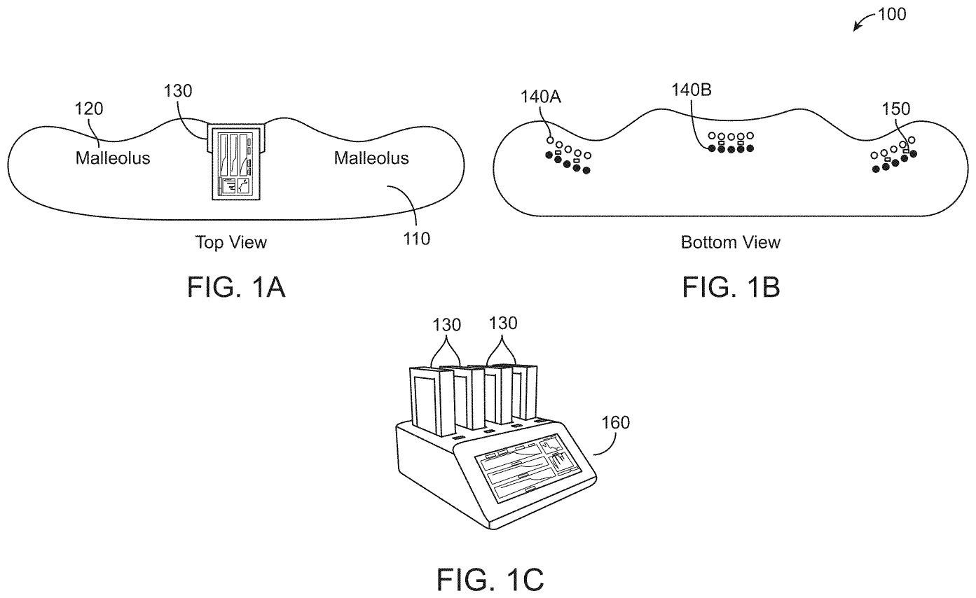

FIG. 1A illustrates the top view of an embodiment of a blood volume monitoring device 100. In various embodiments, non-invasive blood volume or oxygen saturation measurements can be obtained directly and/or indirectly through methods such as optical sensors, photoplethysmography, ultrasound, pressure sensing and/or sound. Measurement data and differences in blood flow volume can be stored, recorded, trended and/or analyzed. The device 100 can be secured fully, partially or not at all around a body part and lay over the skin area of the plurality of predetermined locations. The one or plurality of predetermined locations may be situated on a wrist, neck, chest and/or an ankle of the subject. In some embodiments, base layer is configured to attach, encompass, or mount onto the portion of the subject's body using at least an adhesive, strap, suction, or electromagnetic mechanism. Non-limiting examples of fastening and or securing attachments are straps, adhesive tapes, Velcro and other fastening mechanisms. The device may comprise a base layer 110 which includes the elements of the device. The base layer may comprise a wearable patch. The base layer may be made of flexible and/or biocompatible materials. The wearable patch may be single use (disposable) or multiple use. Markers 120 may indicate one or a plurality of predetermined locations to the proximity of the target site. The circuitry (power and processing unit) may be an integrated part of the device. In some embodiments, the circuitry is detachable from the device. The circuitry may include power source, backing material, communication component, processor, data storage module and other electrical and/or electronic components. The detachable circuitry may be located on a rechargeable link 130. The power source may comprise a battery. The communication component may be a transmitter.

FIG. 1B shows the bottom view of an embodiment of the device 100 which is in contact with the skin or the anatomical part of the subject's body. In one embodiment, the device may comprise a plurality of light sources such as but not limited to light emitting diodes (LEDs) 140A and 140B. In some embodiments, the device may comprise at least three LEDs or three arrays of LEDs. The LEDs may be configured to emit light to penetrate beneath the skin of the subject. The light may be emitted at one wavelength or two different wavelengths or three or more different wavelengths. In some embodiments, the LEDs may emit light at three or more different wavelengths. Nonlimiting examples of the emitting wavelengths of LEDs are red light spectra wavelength between 600 nanometers (nm) and 700 nm, infrared light spectra wavelength between 800 nm and 1000 nm, green light spectra wavelength between about 520 nm and 560 nm and orange light spectra wavelength between about 590 nm and 650 nm. In some embodiments, each LED or a subset of LEDs may emit one wavelength. In other instances, each LED or a subset of LEDs may be augmented to be capable of emitting multiple wavelengths. In some embodiments, the one or a subset of LEDs may emit near infrared light. The LEDs may emit light at different pulsed frequencies.

The device may comprise a plurality of light detector sensors such as photodiodes 150. Each photodiode or a subset of photodiodes may be excited by one or a plurality of wavelengths. In some embodiments, each photodiode or a subset of photodiodes can be excited by the interaction between the plurality of wavelengths emitted by LEDs. In some embodiments, the LEDs may emit light as well as detect the received light from the body of the subject. In some embodiments of the device, the plurality of LEDs and photodiodes may be disposed within the base layer 110. In some cases, the base layer may serve as a substrate for supporting the plurality of LEDs and photodiodes. In some embodiments, the base layer may comprise a plurality of wires or electrical connections for electrically connecting the plurality of LEDs and photodiodes. The spacing between the LED elements may be 1 millimeter (mm), 2, 3, 4, 5, 6, 7, 8, 9 or 10 millimeters (mm) or any value in between said values. The spacing between the LED elements may be larger than 10 mm. The spacing between the LED elements may be smaller than one mm. The spacing between the LED elements and the photodiodes may be 1 millimeter (mm), 2, 3, 4, 5, 6, 7, 8, 9 or 10 millimeters (mm) or any value in between said values. The spacing between the LED elements may be smaller than one mm. The spacing between each sensor array may be 10 mm, 20, 30, 40, 50, 60 or 70 mm or any value in between said values. The spacing between each sensor array may be larger than 70 mm. The spacing between each sensor array may be smaller than 10 mm.

The plurality of LEDs and the photodiodes may be located separately on the device or be integrated into a single unit or component such as a sensor array. The device 100 may be located over one or a plurality of predetermined locations for example in some embodiments, the device may be located over three predetermined locations. In some instances, the device may be located over more than three predetermined locations. In some instances, the device may be located over one or two predetermined locations. The device 100 may comprise one or a plurality of sets of light emitters and detectors depending on the number of predetermined locations. For example, the device may comprise three sets of light emitters and detectors as shown in example of FIG. 1B.

FIG. 1C shows an example of a charging station 160 for the rechargeable links 130. The charging station may be able to charge one or a plurality of rechargeable links. Nonlimiting example of charging station may be a power adapter for charging the battery of the device before or after the usage of the device.

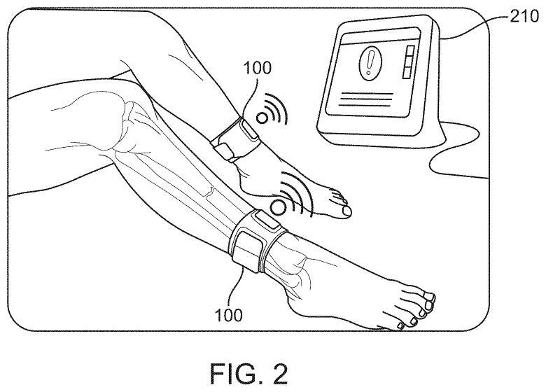

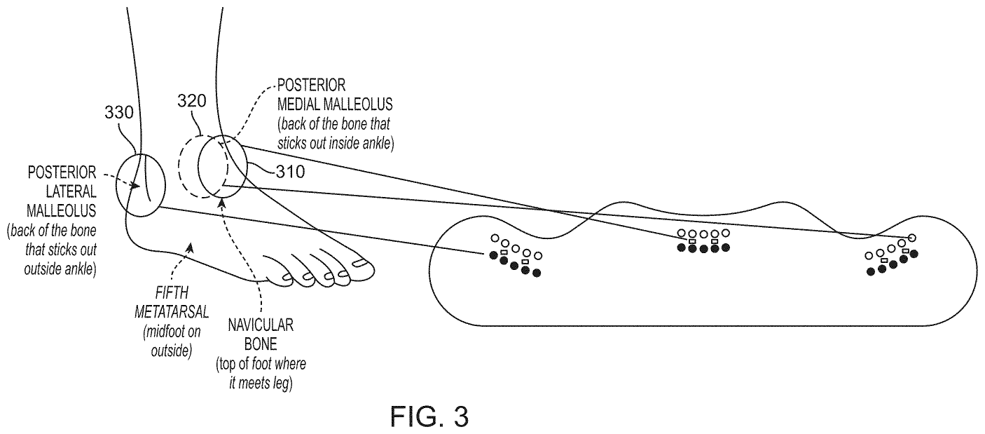

FIG. 2 illustrates an embodiment of the device 100 form factor or patient interface in which the device may be all in one and can be worn on the body. The device may be fixed in place or removable. The device can include a modular system and/or adaptor for mounting. As shown in FIG. 2 a present embodiment of the device can include a device worn on the user's extremity that can be attached and/or removed using a suitable mechanism such as but not limited to clip, Velcro, adhesive, zipper or plastic clamp. FIG. 2 shows an embodiment of the device located on the lower extremity of a subject's body. In some embodiments, the device may be used as a pair in two different sites. The data from one of the paired devices may be used as baseline or reference data and for comparison with the target site data. In some embodiments, the device 100 may be in communication with a remote device 210. The communication may include but is not limited to sending and receiving operational commands such as turning the device 100 on or off, transferring data to a remote storage or server, etc. device 100 may be connected to remote device 210 through wired connections or wirelessly.

The predetermined locations for the device may be situated adjacent to bony structures of the subject's body. The bony structures in human subjects may be selected from a group consisting of medial malleolus, lateral malleolus, spinal column, femur, calcaneus, fibula, radius, ulna, scaphoid and tibia. The predetermined locations may be situated directly above muscular tissue of subject's body.

A device as described herein can be configured to monitor a target site in a subject's body, the device comprising a base layer configured to attach, encompass, or mount onto a portion of the subject's body in proximity to the target site; a plurality of light emitting diodes (LEDs) arranged on or coupled to the base layer to correspond to at least one section of thin tissue on the portion of the subject's body, wherein the plurality of LEDs are configured to emit light signals into at least one section of thin tissue to penetrate the skin of the subject to reach the target site; and a plurality of photodiodes configured to receive reflected light signals associated with the target site, wherein the reflected light signals are analyzed to determine a plurality of physiological parameters comprising at least volumetric blood flow through, at, or in proximity to the target site, and wherein the plurality of physiological parameters are used to monitor a state of the target site.

In some embodiments, the plurality of LEDs and photodiodes may be operably coupled to a processor via wired or wireless communications. The processor may be a part of device 100 or separate from it. In some embodiments, the device 100 further may comprise the processor. In some embodiments, the processor may be removable from the device while the device maintains operability and functionality. In some instances, the processor may be provided separate from the device. In some cases, the processor may be located on a mobile device or an external monitoring unit. In those instances of the disclosure that include processor, the processor can be configured to monitor the state of the target site based on a plurality of measurements taken over a time period, wherein the time period can be on the order of hours, days, weeks, or months. The state of the target site may comprise the health of the target site. The processor may be configured to receive the reflected light signals from the plurality of photodiodes. In some embodiments, the processor may be configured to control the plurality of LEDs to emit light signals at three or more different wavelengths and various pulsed frequencies. In other instances, the processor may be configured to control the plurality of LEDs to emit light signals at two different wavelengths and various pulsed frequencies.

FIG. 3 shows an example of the placement of the device to the proximity of the target sites. Each predefined location may correspond to a palpable pulse. In the example of FIG. 3 predefined location 310 may be to the proximity to the navicular bone. Predefined location 320 may be to the proximity to the posterior medial malleolus. Predefined location 330 may be to the proximity of posterior lateral malleolus. In some embodiments of the device, the one or more predetermined locations may be situated superior to arterial structures or in proximity to arterial structures.

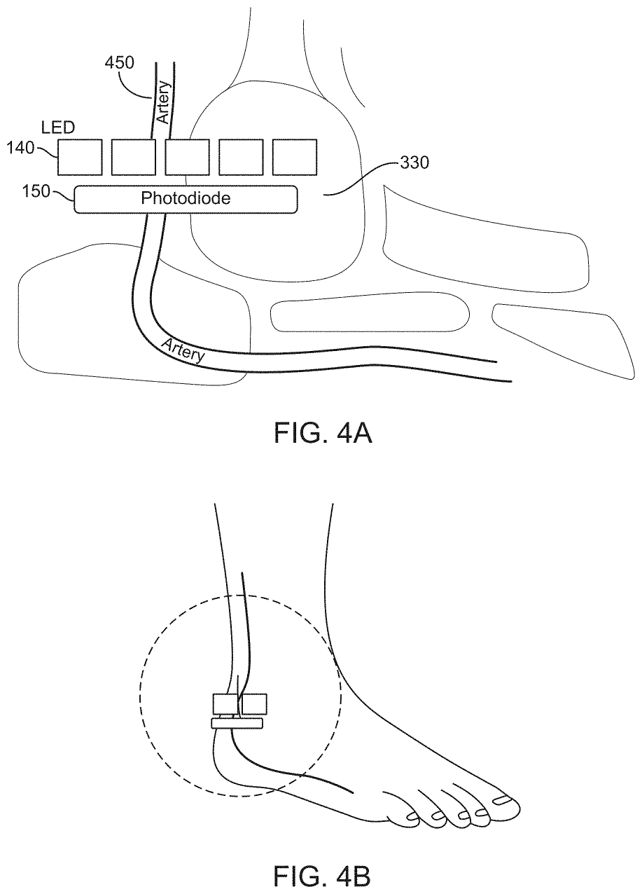

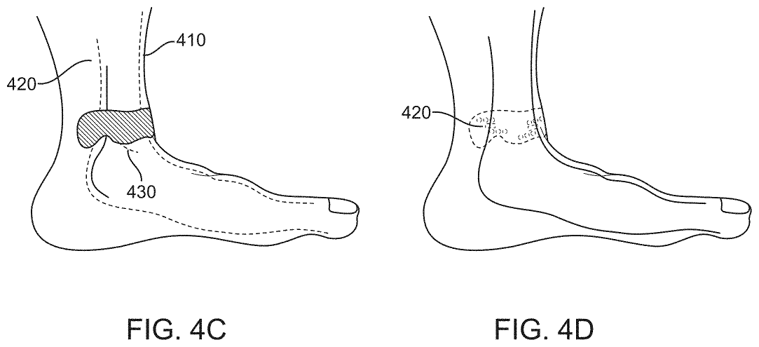

FIG. 4A and FIG. 4B show an example exploded view of location of the sensor arrays including the plurality of LEDs and photodiodes over an example target site such as to the proximity of lateral malleolar artery450 and posterior lateral malleolus bone 330. The plurality of LEDs may allow for detecting target site for different subjects with different anatomy with an accepted margin of error. The emitted light signals may be partially absorbed by the arterial structures, and reflected by the arterial structures as the reflected light signals.

FIG. 4C and FIG. 4D show an example of exploded view of location of the sensor arrays including the plurality of LEDs and photodiodes with regards to the arterial structure. In FIG. 4C the device is in the proximity of anterior tibial artery 410, posterior tibialis artery 420 and malleolar arteries 430. As seen in FIG. 4D, one of the plurality of sensor arrays (LEDs and photodiodes) is located at the predetermined location 420 which in this example is in the proximity to posterior tibialis artery.

One aspect of the present disclosure may comprise a method for noninvasively monitoring a target site in a subject's body, the method comprising attaching a device to a portion of the subject's body in proximity to the target site, wherein the device comprises a base layer, a plurality of light emitting diodes (LEDs) arranged on or coupled to the base layer to correspond to one or more predetermined locations on the portion of the subject's body, and a plurality of photodiodes; using the plurality of LEDs to emit light signals at the one or more predetermined locations penetrating beneath the skin of the subject through, at, or in proximity to the target site; using the plurality of photodiodes to receive reflected light signals associated with the target site; and analyzing the reflected light signals to determine a plurality of physiological parameters comprising at least volumetric blood flow through, at, and in proximity to the target site; and using the plurality of physiological parameters to monitor a state of the target site.

Another aspect of the present disclosure may comprise a method for noninvasively monitoring a target site in a subject's body, the method comprising collecting photoplethysmogram (PPG) signals associated with or in proximity to the target site; analyzing a variation in amplitude of PPG signals over time; using one or more machine learning algorithms to calculate a plurality of physiological parameters comprising at least volumetric blood flow through the target site; and

using the plurality of physiological parameters to monitor a state of the target site, wherein the target site comprises arterial or arteriole structures in an extremity of the subject, and wherein the state is characterized by tissue health through, at, or in proximity to the target site.

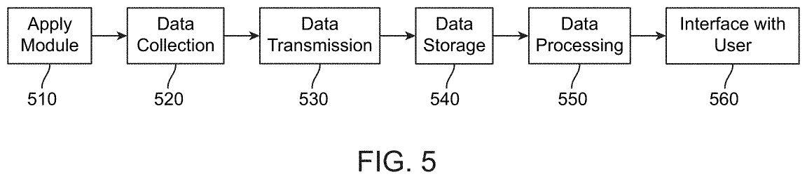

FIG. 5 shows the block diagram of method 500 for capturing and processing the data. In one embodiment, the plurality of LEDs may send data such as light signals and the photodiodes may receive data signals from the proximity of the target site. The light intensity detected by the photodiodes or the amount of light absorbed by the body may be used to calculate a number of physiological parameters. The processor can process data from each photodiode element 150 separately or as a combination. The processor may be a hardware processor such as a central processing unit (CPU), a graphic processing unit (GPU), a general-purpose processing unit, which can be a single core or multi core processor, or a plurality of processors for parallel processing. The processor can be any suitable integrated circuit, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations. Next step may consist of determining a plurality of physiological parameters based on received data. Nonlimiting examples of physiological parameters may include blood oxygen saturation (SPO2) content, intracompartmental pressure (ICP), pulse amplitude, diameter of the arterial structures, blood volume, velocity of the blood within the arteries, heart rate, heart rate variability, respiration rate and blood pressure.

The state of the target site may be determined based on the physiological parameters. In some embodiments the state of target site may be indicated by one or more physiological parameters.

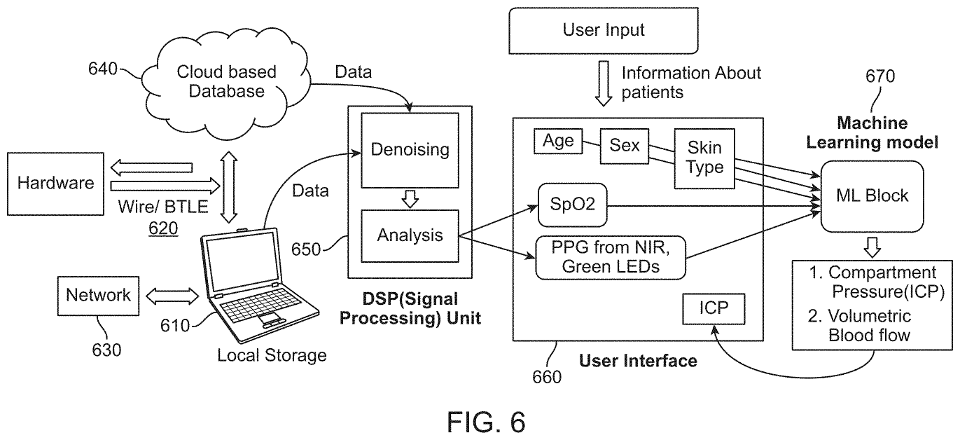

FIG. 6 illustrates an embodiment of the monitoring system wherein the system may consist of one or more data acquisition and storage devices 610 for wired or wireless data transfer 620, including but not limited to Bluetooth, Bluetooth Low Energy (BTLE), cellular and/or wireless local area networking. The data storage device 610 can include but is not limited to metadata. Data can be stored in one or a plurality of databases. The databases can be in local storage system or on remote servers such as cloud servers. The one or more databases 640 may utilize any suitable database techniques. For instance, structured query language (SQL) or “NoSQL” database may be utilized for signal data, raw collected data, training datasets, trained model (e.g., hyper parameters), weighting coefficients, etc. Some of the databases may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used.

The network 630 may establish connections among the components in the monitoring system and a connection to external systems. The network 630 may comprise any combination of local area and/or wide area networks using both wireless and/or wired communication systems. For example, the network 630 may include the Internet, local area network (LAN), as well as mobile telephone networks. In one embodiment, the network 630 may use standard communications technologies and/or protocols. The data exchanged over the network can be represented using technologies and/or formats including data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

Various data operations and manipulations may be applied at the signal processing unit 650 including but not limited to data filtering, denoising, DC filtering and data compression. Other mathematical functions may be applied to filtered data to obtain physiological parameters. Physiological parameters may be displayed in different forms such as tables or simple text or graphs on a graphical user interface (GUI). The user may be able to input data on the GUI such as personal information including age and gender.

In some embodiments, the processor is configured to analyze the reflected light signals using one or more machine learning algorithms to determine the plurality of physiological parameters. The monitoring system may utilize machine learning algorithms for analysis of the data such as extracting intracompartment pressure the target site and volumetric flow and risk assessment and prediction. The example system of FIG. 6 may include a machine learning block 670. The machine learning block 670 may include a training module. The training module may be configured to obtain and manage training datasets. The training datasets may include historical data or human data from pre-clinical studies or simulated data. The training datasets may include data such as but not limited to physiological parameters as well as personal attributes such as age, height, weight, gender, skin melanin content, body mass index (BMI) or medical history. The training module may be configured to train a deep learning network for risk assessment and/or prediction. The training module may comprise a supervised or unsupervised learning method such as, for example, SVM, random forests, clustering algorithms, gradient boosting, logistic regression, or decision trees. The machine learning block may comprise a neural network comprising a convolutional neural network (CNN), recurrent neural network (RNN), long short-term memory RNN (LSTM RNN), dilated CNN, fully connected neural networks, deep generative models or deep restricted Boltzmann machines.

The training module may train a model off-line. Alternatively or additionally, the training module may use real-time data as feedback to refine the model for improvement or continual training.

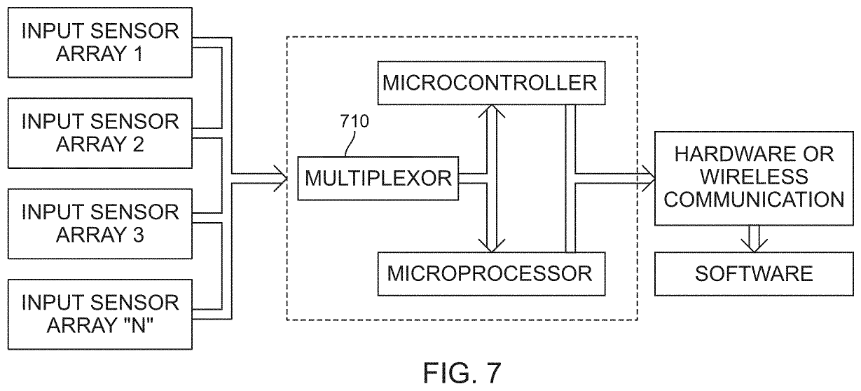

FIG. 7 shows an example of the hardware and software architecture of the monitoring system. In some embodiments, the plurality of LEDs and photodiodes collectively constitute a plurality of sensing channels. The reflected light signals from the plurality of sensing channels may be provided to a multiplexer. Each element of the sensor array may be connected to a data acquisition system, such as digital processing device 650 via interface electronics. The interface may include a switch which sets the sensor array elements for either transmit or receive mode. The position of the switch may be determined by an appropriate computing device, such as digital processing device 650. In the transmit mode, the computing device may receive a signal from the control system instructing one or a plurality of light pulses with specific wavelength(s) to be emitted. The computer may initiate the pulse by sending a signal to a digital-to-analog converter (DAC), which may shape the pulse. The DAC may send the signal to a power amplifier that may generate sufficient power for the pulse. The amplified signal may travel through the switch, to the sensor and into the tissue.

In some embodiments, a transmit system may consist of digital waveform storage, digital-to-analog conversion, linear power amplification to drive the LEDs, and a multiplexer 710 to select the elements(s). The transmit signal may be stored in a memory internal to a computing device. The waveform may be clocked out to a DAC at a clock rate and timing as determined by the programming of the system, as set up by a user. A linear power amplifier may be used to provide an element drive. Multiplexer switches may steer the transmit signal to an element. The transmit system may be capable of generating diagnostic signals for electrical loopback testing and calibration of the analog transmit/receive (T/R) chain.

In some embodiments, in the receive mode, the digital processing device 650 may set the switch accordingly. A sensor array element may detect a signal, which may be sent to a filter via the switch. The filter may remove unwanted information, such as interference, or DC component and may pass the information to a signal conditioner. The signal conditioner may, among other things, convert an analog signal to a digital signal, process the signal to extract useful information from various frequency bands of the signal, and appropriately buffer the information for transmission to the data acquisition system. At the appropriate time, the information may be read out of a buffer by the digital processing device and transmitted to the data acquisition system. In some embodiments, the receive signal conditioning path from the sensor array element consists of a T/R switch, a low noise preamp, low pass filters, and an ADC. The T/R switch may be a biased diode bridge which may block large amplitude transmitted signals, but may allow received signals to pass to the preamplifier. The low noise preamplifier and TGC amplifier may be embodied in a number of commercial integrated circuits. The TGC output stage may drive anti-aliasing low-pass filters to an ADC. The ADC may be multiplexed in groups to allow reduced I/O port usage. The data may be written to standard SDRAM, or similar standard PC RAM to allow for economical data storage.

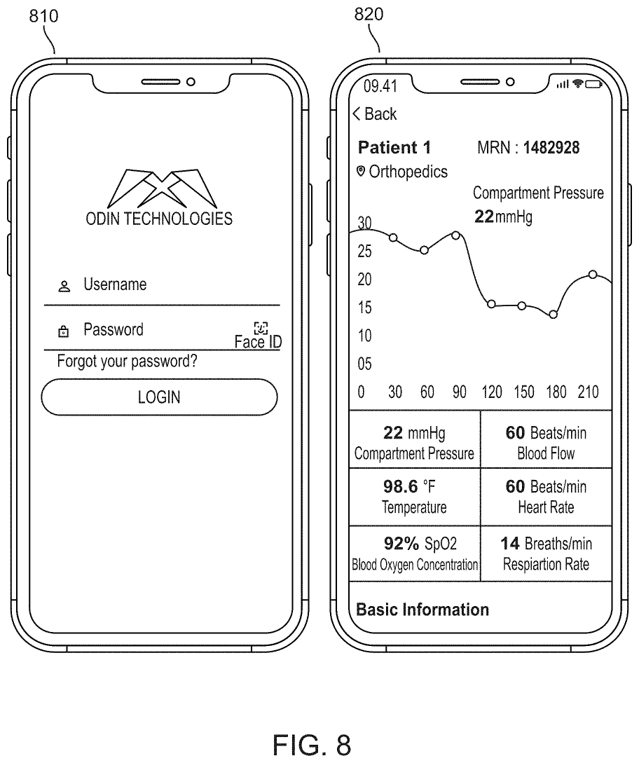

FIG. 8 shows examples of a graphical user interface (GUI). In some embodiments, the plurality of physiological parameters is configured to be displayed on a graphical user interface and/or indicated through visual, auditory, or tactile stimulus. Non-limiting example of a GUI is a toolkit on a smartphone enabling interaction with the patient for purpose of for example including the patient in a study or motivating the patient to comply with the care team requirements. In some embodiments a user may enter data of some activity or a parameter or personal information. For example, window 810 shows that the subject can create an account for privacy and data security reasons. In some embodiments, the user can view different metrics or statistics related to parameters such as but not limited to physiological parameters related to the state of target site. The user may include but is not limited to the patient, physician, health care provider, health care administrator or any other party that may have been given access the user's profile. For example, window 820 shows metrics such as intracompartment pressure, blood pressure, temperature of the target site, blood flow (number of pulses per minute at the target site), respiration rate (number of breaths per minute), blood oxygen concentration (SPO2), basic information about the patient such as age, gender, etc., as well as some graphics related to the state of the target site such as a graph of the variation of compartment pressure during a period of time. Window 820 shows an example of an embodiment wherein a graphical representation can be expanded in full screen to give a better view and contain the information over longer duration of time. In some embodiments, a report can be generated and shown based on the measurements over time. This report can be indicative of the state of the target site. In some embodiments, a notification module may be configured to send one or more alerts or status reports while the target site is being monitored using the plurality of physiological parameters.

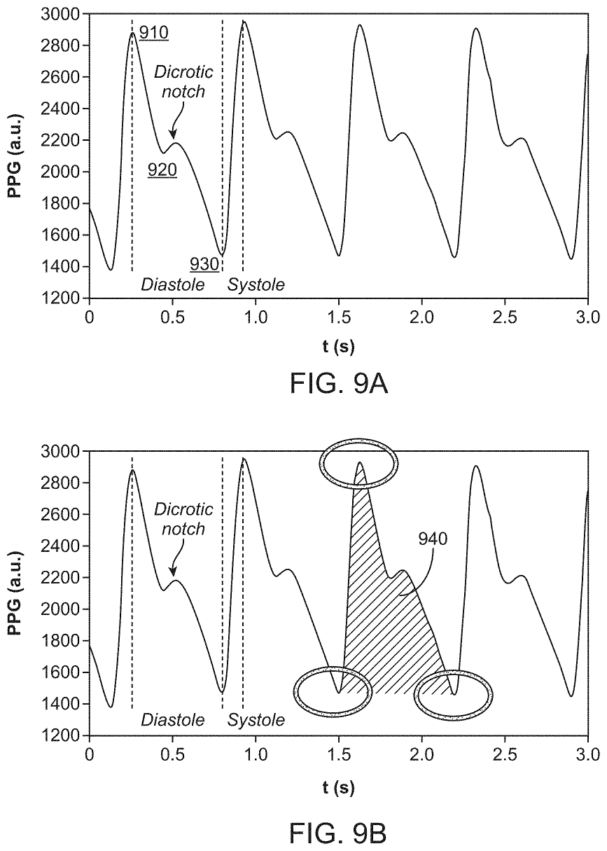

FIG. 9A shows a graph of photoplethysmogram (PPG) signals (y-axis) during a time period. In some embodiments, the reflected light signals may comprise PPG signals. This exemplary graph may be indicative of pulse or may lead to measurement of blood flow in and out of the target site. A few points may be distinguishable on the graph of PPG signals among which are systolic maxima 910, dicrotic notch 920 and diastolic minima 930. In some embodiments, the processor may measure the impulse strength as the area 940 under the curve of PPG signals.

In some embodiments, the digital processing device or the processor may analyze the reflected light signals. The analysis of the reflected light signals may comprise comparing received amplitudes of consecutive PPG signals over different time periods. The time period can be on the order of seconds, minutes, hours, days, weeks, or months. In some embodiments, the amplitude of consecutive PPG signals may be compared by comparing the reflected light from the same photodiode. In other cases, the amplitude of consecutive PPG signals may be compared by comparing the reflected light from different photodiodes.

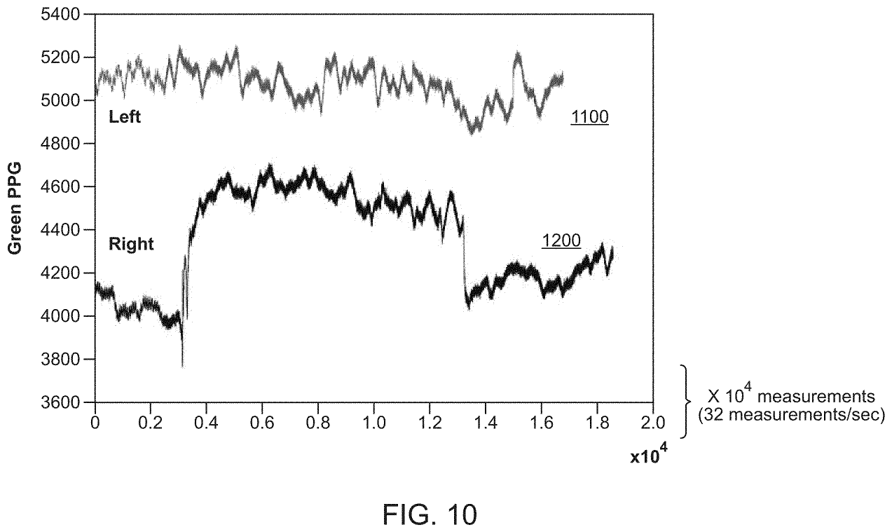

As shown in FIG. 10, the impulse strength of consecutive PPG signals may then be plotted or be gathered in a table for periods of time to indicate the small changes and trends of the impulse over time. In the example of FIG. 10, the impulse measurements are shown for left leg 1100 as well as for right leg 1200. In this example, data is sampled at the rate of 32 samples per second. The comparison between different target sites may indicate the abnormality in one of the target sites.

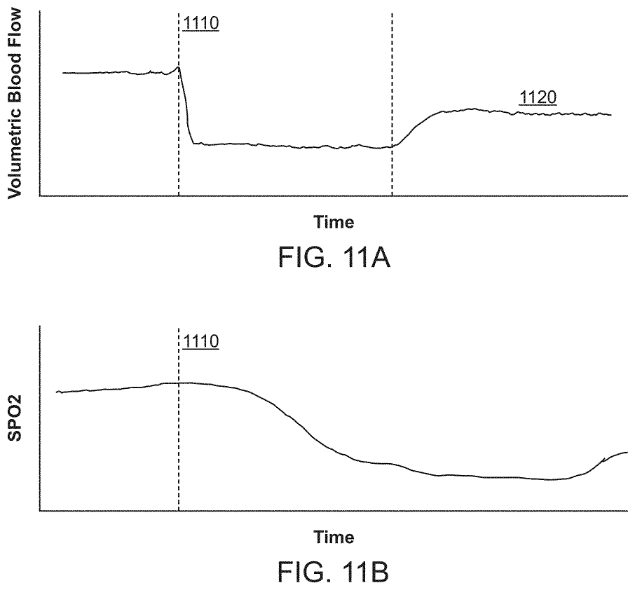

The volumetric blood flow may change in response to stimuli such as regional pressure or application of a tourniquet. FIGS. 11A and B shows examples of variation in volumetric blood flow in time and variation in blood oxygen saturation content (SPO2) in time accordingly. As shown, when a stimulus 1110 is applied such as application of a tourniquet, the volumetric blood flow as well as SPO2 may drop. The removal of stimulus 1120 such as removal of stimulus may cause another change in the volumetric blood flow and SPO2. These changes may also be reflected in the graph of FIG. 10.

The measurement of plurality of physiological parameters such as PPG signals, volumetric blood flow, SPO2, etc., may be used to determine a risk level of a subject for developing a variety of diseases or symptoms such as but not limited to ischemic tissues or compartment syndrome. The physiological parameters may be used to assess whether the subject has perfusion related complications or is at risk of developing perfusion related complications. Machine learning algorithms may allow for analysis of physiological parameters, risk assessment and determination substantially in real time as described elsewhere herein. The plurality of physiological parameters may be used to monitor the progression or regression of any perfusion related complications. In some embodiments, if the risk level is larger than a predetermined threshold the patient and/or the healthcare provider may be alerted in real time.

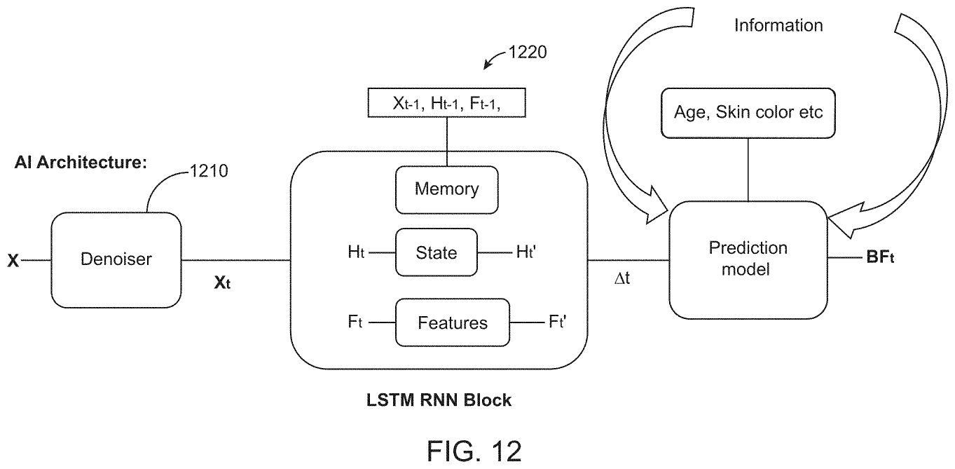

FIG. 12 shows an example of artificial intelligence (AI) architecture. The data such as PPG time series received by the sensor array may be denoised at the denoiser module 1210. The denoising process may improve the signal to noise ratio of the raw PPG signal X(t). At each time step t, denoised data may be used as input and processed at an artificial neural network such as long short-term memory (LSTM) recurrent neural network (RNN) 1220. Various data features may be calculated from the input data including but not limited to heart rate, respiration rate, blood pressure, heart rate variation, SPO2 and amplitude of PPG signal. As the denoised data at time step t, enters feed forwarding LSTM RNN 1220, the states (Hₜ) of machine learning block module and features (Fₜ) may be extracted and derived respectively from the PPG signal. The extracted features as well as the state may be forwardly fed into a LSTM cell via an input gate and may be processed by an activation neuron (not shown in the figure) through computing an activation function and a threshold. The threshold may then retain the past states (or memory) of the computation node (e.g. Xₜ₋₁, Hₜ₋₁, Fₜ₋₁) for the time when the network was processing the PPG signal at the previous time step (e.g. t−1). The feedback of the processing parameters at the previous time step or steps may then be backward-fed through a Peephole to the current processing cell. After processing, the processed signal, state and feature at time step t, may then be forwardly fed through an output gate to a prediction model, where the patient specific information such as age, gender, skin tone, etc. may be incorporated to the model to fine tune the performance of the model.

The prediction model may be able to predict the probability or likelihood of an abnormality at the target site such as perfusion related complications. It will be understood that LSTM RNN 1220 is provided as an illustrative but not limiting example. Any other suitable processing technique such as but not limited to fuzzy logic techniques, Markov models, state machines, Bayesian logic techniques, evolutionary or genetic algorithms or a combination thereof may be used.

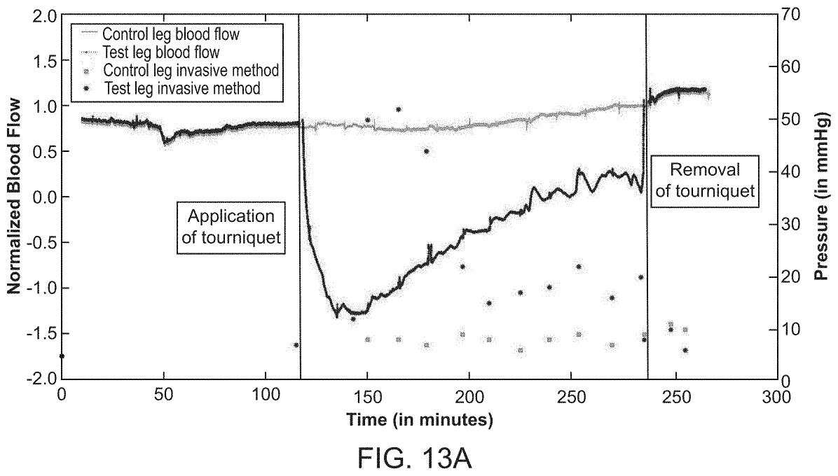

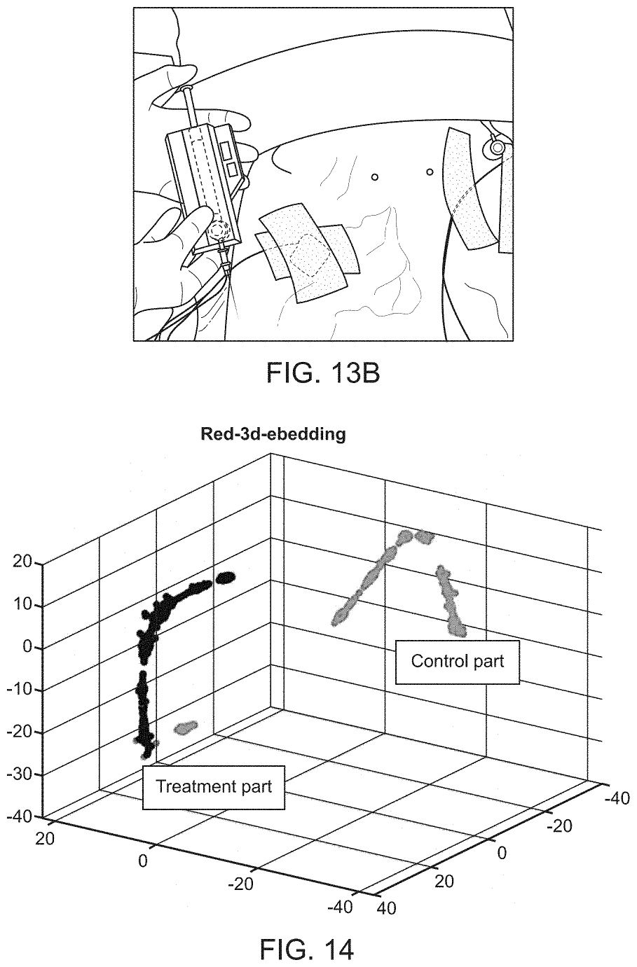

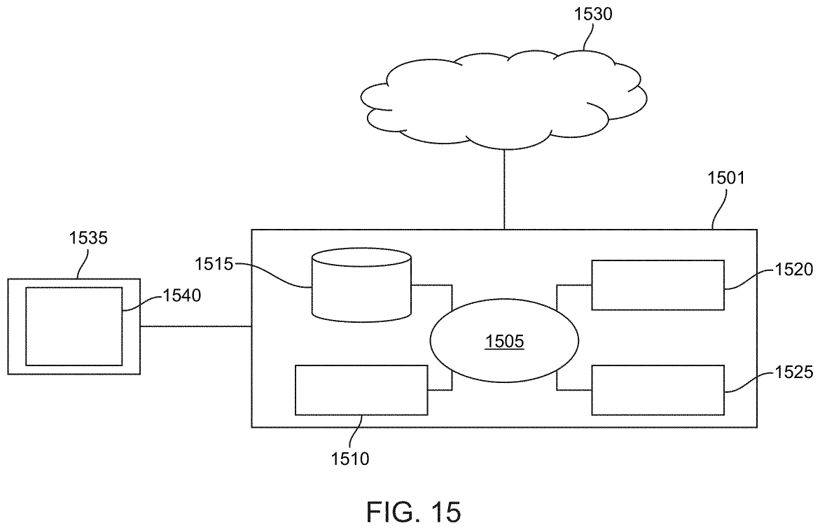

FIG. 13A shows a comparison between the results from the present method and an example invasive method of measurement as shown in FIG. 13B. Normalized blood flow is shown for control leg and test leg in both methods. As shown, the method of present disclosure presents a continuous measurement of blood flow which allows for observation of subtle changes over short durations as well as larger changes and/or variations occurring over longer periods of time. The repetition of invasive measurement technique adds discomfort for patient while the method of present disclosure is noninvasive and capable of continuous measurements. FIG. 14 shows an example of data collected from the device of present disclosure. As shown in this example, data from treatment part is spatially congregated (arc on the left) in a reduced dimensional hyper-space (or phase space e.g. a 3D space in figure) and distinctively segregated from the data collected from control part (i.e. arc on the right). There may be substantial signal differences (for example in PPG signal) between affected and unaffected extremities. Compounding these distinctions in PPG signals and their phase space decomposition, it may be possible to define a linear separator (i.e. a linear plane in the reduced dimensional phase space) to classify between the high-risk signal sequence (i.e. arc on the left) and the low-risk signal sequence (i.e. arc on the right).

The present disclosure provides computer systems that are programmed to implement methods of the disclosure. FIG. 15 shows a computer system 11501 that is programmed or otherwise configured to analyze the reflected light signals to determine a plurality of parameters comprising at least volumetric blood flow. The computer system 11501 can be configured to analyze data and use machine learning algorithms to assess the risk level for developing or progression of various complications such as but not limited to compartment syndrome or tissue ischemia as described elsewhere herein. Alternatively or additionally, the computer system 11501 can be configured to run the application described in FIG. 8. The computer system 11501 can be an electronic device of a user or a computer system that is remotely located with respect to the electronic device. The electronic device can be a mobile electronic device.

The computer system 11501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently pro-gram or otherwise configure the CPU 1505 to implement methods of the present disclosure. Examples of operations performed by the CPU 505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user (e.g., send information such as measurement data or other data). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus with-in a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, the generated 3D proportionately scaled model of the user. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, analyze the reflected light signals such as PPG signals and measure intracompartment pressure. The algorithms may use machine learning or artificial intelligence methods for signals analysis and making the measurements.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for non-invasive monitoring of a target site in a subject's body, the device comprising:
a base layer configured to attach, encompass, or mount onto a portion of the subject's body in proximity to the target site;
a plurality of light emitting diodes (LEDs) arranged on or coupled to the base layer corresponding to one or more predetermined locations on the portion of the subject's body, wherein the plurality of LEDs is configured to emit light signals at the one or more predetermined locations penetrating beneath the skin of the subject to the target site; and
a plurality of photodiodes configured to receive reflected light signals associated with the target site,
wherein the reflected light signals are analyzed to determine a plurality of physiological parameters comprising at least volumetric blood flow through, at, or in proximity to the target site, and
wherein the plurality of physiological parameters is used to monitor a state of the target site.

2. The device of claim 1, wherein the target site comprises muscle tissue, arterial structures, and/or venous structures in an extremity of the subject, and wherein the extremity includes an upper extremity and/or a lower extremity.

3. The device of claim 1, wherein the state is characterized by tissue health at the target site.

4. The device of claim 1, wherein the light signals are emitted at three or more different wavelengths.

5. The device of claim 1, wherein the one or more predetermined locations are situated adjacent to bony structures of the subject's body, and wherein the bony structures are selected from the group consisting of medial malleolus, lateral malleolus, spinal column, femur, calcaneus, fibula, radius, ulna, scaphoid, and tibia of the subject, are situated directly above muscular tissue of the subject's body, or are situated on a wrist, neck, chest, and/or an ankle of the subject.

6. The device of claim 1, wherein the plurality of LEDs and photodiodes are operably coupled to a processor via wired or wireless communications.

7. The device of claim 6, further comprising the processor, wherein said processor is configured to receive the reflected light signals from the plurality of photodiodes.

8. The device of claim 6, further comprising the processor, wherein said processor is configured to control the plurality of LEDs to emit the light signals at two or more different wavelengths and various pulsed frequencies.

9. The device of claim 6, further comprising the processor, wherein said processor is configured to analyze the reflected light signals to determine the plurality of physiological parameters.

10. The device of claim 9, wherein the plurality of physiological parameters further comprises blood oxygen saturation (SPO₂) content.

11. The device of claim 1, wherein the plurality of physiological parameters is used to determine a risk level of the subject for developing ischemic tissues, a risk level of the subject for developing compartment syndrome substantially in real time, whether the subject has perfusion related complications, or whether the subject is at risk of developing perfusion related complications.

12. The device of claim 1, wherein an amount of light absorbed by the body associated with the reflected light signals is analyzed by a processor to generate a plot comprising photoplethysmogram (PPG) signals.

13. The device of claim 1, wherein the plurality of physiological parameters further comprises a calculation for a diameter of an arterial structure.

14. The device of claim 1, wherein the one or more predetermined locations are further situated in proximity to arterial structures.

15. The device of claim 6, further comprising the processor, wherein said processor is configured to analyze the reflected light signals using one or more machine learning algorithms to determine the plurality of physiological parameters.

16. The device of claim 15, wherein the plurality of LEDS and photodiodes collectively constitute a plurality of sensing channels, wherein the reflected light signals from the plurality of sensing channels are provided to a multiplexer.

17. The device of claim 16, wherein the device comprises the multiplexer.

18. The device of claim 1, wherein the base layer is configured to attach, encompass, or mount onto the portion of the subject's body using at least an adhesive, strap, suction, or electromagnetic mechanism.

19. The device of claim 1, wherein the plurality of physiological parameters is configured to be displayed on a graphical user interface and/or indicated through visual, auditory, or tactile stimulus.

20. The device of claim 19, wherein said device is operably coupled to a notification module that is configured to send one or more alerts or status reports while the target site is being monitored using the plurality of physiological parameters.
